(19) United States
(12) Patent Application Publication
(10) Pub. No.: US 2007/0166185 A1
(43) Pub. Date: Jul. 19, 2007

(54) Method for dispensing liquid fragrances and device for carrying out the method

(75) Inventor: Frank Bartels, Hattingen (DE)

Correspondence Address: HENRY M FEIEREISEN, LLC, 350 FIFTH AVENUE, SUITE 4714, NEW YORK, NY, 10118, US

(73) Assignee: International Flavors & Fragrances, Inc., New York, NY (US)

(21) Appl. No.: 10/566,414

(22) PCT Filed: Jul. 28, 2004
(86) PCT No.: PCT/DE04/01680

§ 371 Date: Jan. 30, 2007

(30) Foreign Application Priority Data
Jul. 28, 2003 (DE) 103 34 591.4

(51) Int. Cl. A61L 9/04; A61L 9/12

(52) U.S. Cl. 422/5; 422/123

(57) ABSTRACT

The invention relates to a method for the distribution of liquid fragrances, using a device with at least one inlet line (2), for supply of the fragrance to at least one dispenser unit (4), where the supplied fragrance is converted to an aerosol by the action of an electrical charge. A high voltage unit (5), connected to the dispenser unit (4), provides the fragrance with an electrical charge. A controller (6) and at least one stop and adjustment body (3), connected to the controller, serves to block the supply line. The stop and adjustment body (3) and the high voltage unit (5) are operated with a relative timeshift such that the amount of fragrance in the supply line (2), between the stop and adjustment body (3) and the dispenser unit (4), is reduced. The stop and adjustment body (3) is preferably a micropump. The discharge of the fragrance occurs by means of the electrospray technique.

### BACKGROUND OF THE INVENTION

The invention relates to a method for dispensing liquid fragrances, and a device for carrying out the method.

Fragrances and combinations of fragrances released into a room are used not only for improving the climate in a room, but also for biological treatment and for communications, for example to enhance a person's attention. It is known that the attention can be enhanced by simultaneously employing audio-visual and olfactory media. For example, fragrances can be released synchronously with audio-visual presentations and distributed through the ambient air as a carrier.

It is known to convert aromatic essences and other liquids into the gas phase in various ways. The basic physical principles are atomization and evaporation. Evaporation is understood as a gradual transformation of a liquid into a gaseous state. The molecules of each liquid are always in motion between the liquid and the gaseous state. The average velocity increases with increasing temperature. The warmer the liquid, the greater the vapor pressure, i.e., the more liquid evaporates. When the vapor pressure is equal to the ambient air pressure, the liquid begins to boil. Because some molecules are always noticeably faster than others, they can escape from the liquid as a gas. Disadvantageously, the composition of the fragrances can change over time due to evaporation. Fragrances typically consist of a mixture of components having different volatility. Highly volatile fragrances determine the initial scent, whereas scents with low volatility determine the after-scent or basic scent of a fragrance. Changes of the scent over time are normal; however, each fragrance composition should in each phase maintain the same basic character. Evaporation can accelerate changes in the scent, so that the first noticeable initial scent of the fragrance composition also disappears first, whereas less volatile components of the fragrance composition linger. This can have a detrimental effect on the scent, so that fragrances deployed in a room and intended to improve communication ca quickly lose their effect. This effect is enhanced by the increased thermal stress on the fragrance. Although greater volume flows can be produced by evaporation at higher temperatures, the harmony of the fragrance composition frequently suffers.

Alternative to evaporation, the fragrance can also be atomized, whereby a finely distributed mist consisting of a large number of small droplets is generated. The droplets can be formed under pressure, whereby the fragrance is pressurized in a local pressure vessel, for example, by a propellant. It is also known to use piezoelectric actuators for releasing the fragrance, which push a stream of the liquid through individual nozzles.

In conventional arrangements for supplying fragrances, the fragrance can also be transported by a capillary effect. However, with the capillary effect only a certain quantity of the fragrance is in contact with the atmosphere. The initial scent of the fragrance continuously evaporates across the contact surface, so that the scent can change uncontrollably over time.

DE 692 32 096 C2 discloses a device for generating electrostatically charged aerosols and/or vapors, wherein a porous capillary unit with several fibers transports a fragrance from a reservoir to a delivery unit. The fluid is hereby electrostatically charged by a high-voltage DC source and can be dispensed in the form of aerosols and/or vapors from the free tips of the capillary unit. The fluid is transported continuously by capillary action to the upper end of the capillary unit acting as a wick, even if no voltage is applied. The material properties of the capillary unit have a significant impact on the efficiency of the vapor and/or aerosol release at any voltage. The porosity, the electric conductivity and the form of the materials determine the vapor-aerosol-ratio as well as the quantity of the generated air ions. In particular, the individual fragrances evaporate more or less completely, depending on the volatility of the employed fragrances, when very long capillaries are arranged between an optional fluid pump and the discharge end of the capillary unit. This makes it relatively difficult to control of the release of vapors or aerosols.

Based on this concept, it is an object of the invention to provide an easily controllable method for distributing liquid fragrances, wherein a control fluid volume is transformed into an aerosol, while eliminating the uncontrolled formation of droplets on the delivery unit and significantly reducing the possibility of volatile fragrance components to evaporate prematurely.

### SUMMARY OF THE INVENTION

It is another object of the invention to provide a device which can be used to readily implement the method.

The first object is solved with a method for dispensing liquid fragrances, by using a device with at least one supply line for supplying the fragrance to at least one delivery unit, wherein the supplied fragrance is converted to an aerosol by applying an electric charge, a high-voltage unit connected to the delivery unit for applying the electric charge to the fragrance, a controller, and at least one shutoff and actuating element connected with the controller for shutting off the supply line, wherein the shutoff and actuating element and the high-voltage unit are activated with a time offset relative to each other, thereby reducing a quantity of the fragrance disposed inside the supply line between the shutoff and actuating element and the delivery unit.

According to the method of the invention, the shutoff and actuating element and the high-voltage unit are activated with a time offset relative to each other, thereby advantageously reducing the quantity of the fragrance disposed inside the supply line between the shutoff and actuating element and the delivery. Reducing to the quantity in the context of the invention is not to be understood as “condensing” a fluid by supplying thermal energy, but rather as a controlled atomization of the residual quantity by applying the electrostatic spraying process (electro spray). With the electro spray process, the particle size of the generated aerosols is advantageously very uniform and, more importantly, very small. The particle size can be smaller than a micrometer. The highly charged droplets with the same polarity emerging from a capillary tip of the delivery unit repel each other and form an extremely fine mist which is visible only within a small distance from the delivery unit. Already after several centimeters, the fragrance mist can no longer be recognized by the human eye, because it has been converted into the gas phase. The electro spray process can be used to controllably dispense very small quantities of a fragrance.

With this process, the quantity of a fragrance can be reduced to a point where the fragrance is completely released in the region of the delivery unit, so that the fragrance is any longer in contact with the atmosphere. This condition can be achieved either when the device begins to operate or after the fragrance is no longer delivered. In the first situation, the fragrance is distributed by first activating the high-voltage unit and then opening the shutoff and actuating element. This prevents the uncontrolled growth of droplets on the delivery unit which would have to be removed later when the high-voltage unit is switched on. If the high-voltage unit were to be switched on only later, then the droplets jet emerging from the delivery unit would have to penetrate and entrain the adhering droplet. This would cause droplets of different sizes which could produce different scents. If the droplets are very large, these could deposit, due to gravity, in direct vicinity of the delivery unit, so that the fragrance could evaporate entail uncontrollably even after the fragrance has been dispensed. However, if the high-voltage unit is switched on first and the shutoff and actuation element is opened later, then the volume flow of the fragrance at the delivery unit is initially smaller than the volume flow at the shutoff and actuation element, so that the aerosols can be dispensed with a controlled and metered droplet size and quantity.

If the distribution of the fragrance is temporarily interrupted or completely terminated, then the shutoff and actuator element must be closed first, so as to disrupt the supply of the fragrance to the delivery unit. According to the features of claim 3, the high-voltage unit is deactivated only after the shutoff and actuation element or the supply line have been closed. This time-sequential order ensures that the residual quantity of the fragrance remaining between the shutoff and actuation element and the delivery unit is actively delivered, so that this longitudinal section of the supply line is emptied almost completely. The shutoff and actuation element must therefore remain closed until a new delivery of a fragrance to prevent fragrance from being uncontrollably transported to the delivery unit by capillary action.

The advantages of the method of the invention become particularly apparent, when the device delivers several different fragrances simultaneously or with a time delay, for example, for improving or intensifying communication by dispensing scents. The intensity of the scent essentially depends on the released quantity of the fragrance, whereas the communication efficiency depends on the type of the scent and the sequential order in which the scents are released. A precise control and adjustment of the device of the invention is therefore quite important.

In certain applications, a number of different fragrances may be released simultaneously, whereby the intended olfactory effect is attained only by the superposition of the scents. This requires that the fragrances are in harmony with each other and that the individual quantities of the fragrances are exactly matched to each other.

Alternatively, a particular “scent history” can be narrated by delivering individual fragrances with a time offset, without superimposing the individual fragrances. Superposition and mixing of fragrances should be completely prevented, if the sequentially delivered fragrances do not harmonize with each other. This is accomplished with the inventive method by reducing the residual quantities of the initially dispensed fragrance as much as possible, so that even the less volatile base scent of this fragrance is completely eliminated and is not superimposed on the initial scent of the next dispensed fragrance.

The invention has also proven advantageous when different fragrances are dispensed by a single delivery unit. For example, when a first fragrance is stored in an exchangeable reservoir which is releasably associated with the device, but exchanged later, when empty, against a reservoir containing another fragrance, then the scents the new fragrance should be delivered instantaneously after the exchange, without mixing with residual quantities of the previous fragrance.

According to another feature of the invention, an electric high voltage in a range of between 0.5 kV and 25 kV is applied to the delivery unit. Preferably, the applied electric high-voltage is in a range of between 1.5 kV and 6 kV. The high voltage can be generated in a small space by a high-voltage cascade. Because the high voltage can be generate with a small input voltage, a corresponding device can operate on batteries, i.e., independent from the power mains.

Advantageously, the level of the electric high voltage is maintained constant, while the volume flow of the fragrance is controlled by the shutoff and actuating element. This arrangement can significantly reduce the complexity of the controller in the employed device, because the delivered quantity is controlled only by the position of the shutoff and actuating element. The electric high voltage needs only be switched on and off with a time offset, which has no effect on the delivered quantity. The high voltage can be a DC voltage, and its polarity can change between two spray events.

According to another feature of the invention, the volume flow can be changed with a micropump. Micropumps are known in the art. They can be made of plastic, in particular polycarbonate, and can be actuated by a piezo-ceramic actuator operating on a membrane. The feed rate can be regulated by controlling the amplitude of the piezo-ceramic actuator. Micropumps can be easily integrated in fluid systems and can be manufactured cost-effectively.

Advantageously, the maximum feed volume of the micropump can be adjusted to be smaller than or equal to the delivery capacity of the delivery unit. In this way, the delivery unit can safely accommodate the volume flow conveyed by the micropump, thus achieving constant droplet size and homogeneous atomization of the fragrance.

According to another feature of the invention, each delivery unit is supplied with a different fragrance, wherein the fragrances are separately converted into aerosols by timing or volume control. In this method, different fragrances can be mixed by employing a time and/or volume-dependent control and dispensed in a predetermined temporal sequence. Delivery of the scent can be controlled by a program, for example in wave-form. A corresponding device can be flexibly employed and deliver several fragrances simultaneously. With the electro spray process, even the smallest quantities of scented oils and concentrated fragrances can be controllably and efficiently metered. These devices also have a small installed size and can be controlled by a microprocessor in the controller. Alternatively, within the context of the invention, interfaces to an external controller can also be provided, either to program the internal controller of the employed device or to actively communicate between the external controller and the controller in the device.

The device can be controlled in a continuous automatic mode. However, manual intervention is possible. For example, different automatic modes can be selected manually. Moreover, a user can intervene to select different intensity levels for delivering the fragrance, which can be desirable for adapting to rooms of different size. A high intensity level corresponds to a large delivered volume per unit time. The user should also be able to release with the device one or several fragrances immediately. This can be achieved by providing on the device suitable operating controls, for example one or more pushbuttons.

The service life of the employed device depends, in particular, on the quantity of a fragrance available within a reservoir.

In one advantageous embodiment, a minimal operating time of 45 days under battery power is desired, with approximately 10 to 20 milliliter fragrance delivered to during that time. The delivered quantity depends strongly on the application and the number of delivery units of the employed device. For example, in the context of the invention, a first fragrance may be delivered by a first delivery unit in, for example, five individual blasts, and a second fragrance is delivered immediately thereafter, and optionally additional fragrances. In another operating mode, the delivery of the individual fragrances could be spaced apart by, for example 8 to 10 seconds, to prevent the fragrances from mixing immediately and to space the stimulation by new scents apart. After delivering a sequence of different fragrances with or without interruptions, other fragrances can again be delivered after a pause, either in the same order or in a different order. The pause can last for several seconds; however, depending on the desired delivered quantity, pauses lasting several minutes can also be contemplated. These pauses directly impact the delivered quantity, so that according to the invention the length of the pauses can be continuously adjusted by a manual operating means, without adding complexity to the controller for the shutoff and actuation elements.

According to another advantageous measure, the fragrance can be withdrawn via the supply line from an exchangeable fragrance reservoir having a flexible casing. The fragrance is received in the flexible casing without contact with the atmosphere, so that evaporation of the initial scent of the fragrance can be prevented already in the region of the fragrance reservoir. The total quantity of fragrance is transported from the fragrance reservoir though the supply line and the shutoff and actuation element to the delivery unit without contact with the atmosphere, so that the scent does not change until atomization. The fragrance reservoir can have an indicator for the fill level or an indication when the fragrance reservoir will be totally empty. In the simplest embodiment, the flexible casing has at least one partially transparent region with a scale so that the fill level can be read directly.

Depending on the particular embodiment, the flexible casing can be pressurized so that the fragrance reservoir can be completely emptied and the volume flow can be supplied to the shutoff and actuation element under a minimum pressure. In this configuration, a flow valve controlling the flow can be installed as a shutoff and actuation element, without requiring additional pumping means. The shutoff and actuation element must be closed in its rest position and opened partially or fully only for delivering the fragrance. With a micropump, the fragrance can be adequately withdrawn from the fragrance reservoir by suction. The deformation of the flexible casing under ambient pressure compensates for the decreasing volume in the fragrance reservoir, so that the fragrance reservoir can be almost completely emptied if the casing is flexible enough. The withdrawal opening of the fragrance reservoir is preferably located in the natural flow direction of the fragrance, i.e., at the bottom.

In principle, the shutoff and actuation element can be part of the micropump. For example, the shutoff and actuation element can be a one-way valve disposed in the micropump which only allows flow in one direction. Actuation and/or control are accomplished by controlling the micropump which directly affects the volume flow. It will be understood that additional shutoff and actuation elements can be provided and connected either upstream or downstream of the micropump.

In the context of the present invention, a central pumping unit, in particular a central micropump, can be connected with a delivery unit, wherein the pumping unit can have several supply lines, with each of the supply lines supplying a different fragrance. The supply lines can have different cross-sections so that different quantities of fragrance can be suctioned by the suction effect of the pumping unit, in particular the micropump, either directly into the pumping unit or supplied to an upstream mixing chamber, where the fragrances are then mixed and delivered as a combination of fragrances. One-way valves in the individual supply lines prevent undesirable mixing when the pump unit is deactivated. Alternatively, separate shutoff and actuation elements can be provided in the individual supply lines to separately control the respective supply of the fragrance to the central pumping unit. A corresponding device should, of course, only be used if the superimposed fragrances harmonize with each other. This can be achieved, for example, by mounting certain fragrance reservoirs only in certain positions on the device, which can prevent an unintentional interchange with undesirable consequences for the fragrance composition.

In the context of the present invention, individual fragrance reservoirs can also be coded, with the code being read by a suitable reading unit when the reservoir is attached to the device. The reading unit transmits the read information to the controller, which controls the dose of the newly added fragrance reservoir based on this information. In this way, the dose can be adapted to the fragrances of the already existing fragrance reservoirs. In the employed device, the controller can also react to a change in the fragrance supply, for example, when a fragrance reservoir is empty, by either providing a corresponding signal and/or modifying the quantities of the various fragrances. The remaining fragrances are hereby combined to a new fragrance combination with a new mixing ratio so as to compensate for the temporary loss of fragrance and still have a pleasant scent.

According to another aspect of the invention, a device for carrying of the method includes at least one supply line for supplying the fragrance to at least one delivery unit, where the supplied fragrance is converted to an aerosol by applying an electric charge. The device further includes a high-voltage unit connected to the delivery unit for applying the electric charge to the fragrance, a controller, and at least one shutoff and actuating element connected with the controller for shutting off the supply line. A micropump is also provided which affects the volume flow of the fragrance. The micropump is sized so that the maximal delivery volume of the micropump is less than the maximum delivery capacity of the delivery unit. The quantity of fragrance disposed inside the supply line between the shutoff and actuating element and the delivery unit can be reduced by activating the high-voltage unit and the micropump with a time offset. The shutoff and actuation element of this device is preferably a component of the micropump, so that the delivery unit still operates for a certain time after the micropump is shut off, thereby reducing the quantity of fragrance remaining between the micropump and/or the shutoff and actuation element and the delivery unit. This reduction is achieved, as described above by the electro spray method.

The device of the invention can have several separate delivery units, each with a dedicated supply line and a dedicated micropump, so that a separate fragrance is delivered through each supply line. A single micropump can in principle supply several delivery units connected in parallel, if a particularly high delivery rate of the fragrance is desired. Conversely, several supply lines can be connected to a common micropump. A different fragrance is supplied through each of the various supply lines, and the combined fragrances are then converted to an aerosol through a common delivery unit.

Because the micropumps are quite small components, they can be arranged with considerable flexibility. Most of the space in the device of the invention is expected to be taken up by the fragrance reservoirs and by the device that produces an airflow, for example, a fan. The respective fragrance is atomized in the airflow produced by the fan.

### BRIEF DESCRIPTION OF THE DRAWING

Exemplary embodiments of the invention will now be described with reference to the schematic drawings.

FIG. 1 shows schematically the configuration of a device for carrying out the method for distributing liquids fragrances;

FIG. 2 shows a diagram illustrating the switching times of the delivery unit and the shutoff and actuation element;

FIG. 3 shows a first exemplary embodiment of a device for carrying out the method;

FIG. 4 shows another exemplary embodiment of a device for carrying out the method;

FIG. 5 shows a device for carrying out the method with several delivery units;

FIG. 6 shows a device for carrying out the method with several reservoirs;

FIG. 7 shows a device for carrying out the method with several pumps and reservoirs connected in parallel;

FIG. 8 shows an embodiment with additional shutoff and actuation elements and several delivery units; and

FIG. 9 shows an embodiment similar to that of FIG. 8, however with only a single delivery unit.

### DETAILED DESCRIPTION OF PREFERRED EMBODIMENTS

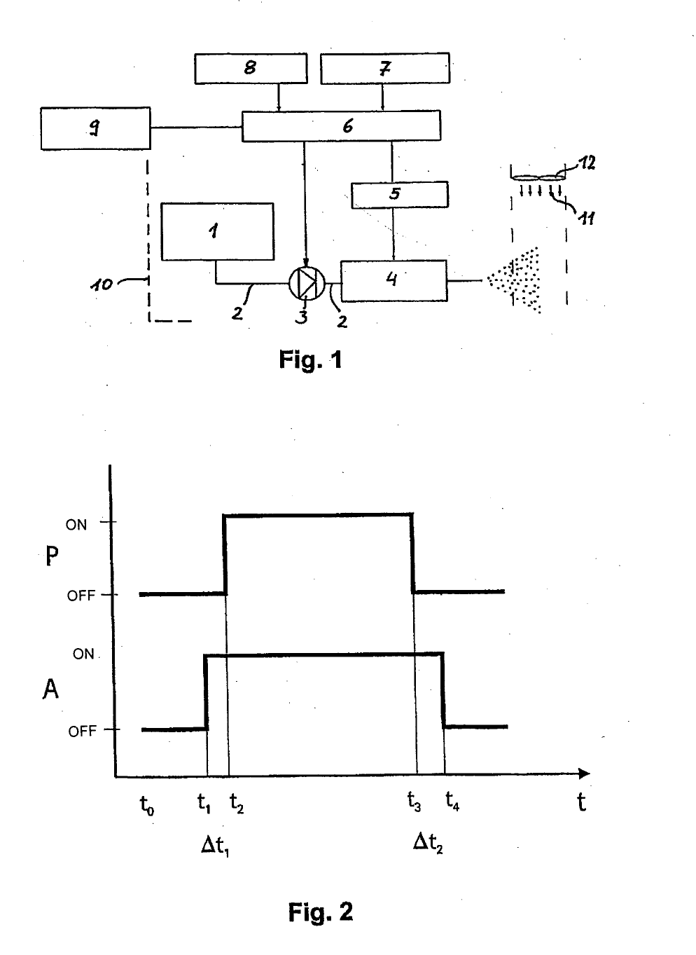

FIG. 1 shows a fragrance reservoir, indicated with reference symbol 1, for storing a fragrance. The fragrance is supplied via a supply line 2 to a micropump 3, which at the same time operates as a shutoff and actuation element. The micropump 3 transports the fragrance to the delivery unit 4, where the fragrance is converted into an aerosol through application of an electric charge. A high-voltage unit 5 connected to the delivery unit 4 applies the electric charge to the fragrance. A DC voltage of between 0.5 kV and 25 kV is applied. The high-voltage unit 5 and the micropump 3 are controlled by a controller 6. Optionally, the controller includes an interface 7 for manual intervention in the atomization of the fragrance and a second interface 8 which can be coupled to an external controller or programming means. An energy source 9 supplying electric energy to the device can be a battery which can be integrated in a (schematically shown) housing 10. Alternatively, electric energy can be supplied by an external source. The delivery unit 4 delivers the fragrance as a fine mist in an airflow 11, which is generated by airflow generating means 12. These means can also be arranged inside the housing 10. Alternatively, these means can also be a component of a climate control.

FIG. 2 shows the time dependence of the activation of a delivery unit A and a micropump P. At time T0, neither the delivery unit A nor the micropump P are activated, i.e., the volume flow in the supply line 2 is zero. At time T1, i.e., when the electro spray process commences, the delivery unit is activated first. The high-voltage unit 5 generates a high-voltage and applies the high-voltage to the delivery unit A. This corresponds to a change in the switching state of the delivery unit A from “OFF” to “ON”. The volume flow is still zero at that time. The micropump is switched on only after a time difference ΔT1, i.e., at time T2=T1+ΔT1, causing the fragrance to flow through the supply line of the delivery unit A. The spray process remains active as long as fragrance is supplied to the delivery unit A. The micropump P is switched off at time T3, while the delivery unit A remains activated. The delivery unit A is finally switched off at time T4=T3+ΔT2. By allowing for the differences ΔT1 and ΔT2, fragrance is not delivered uncontrollably. Switching the micropump P off early prevents the formation of large droplets on the delivery unit.

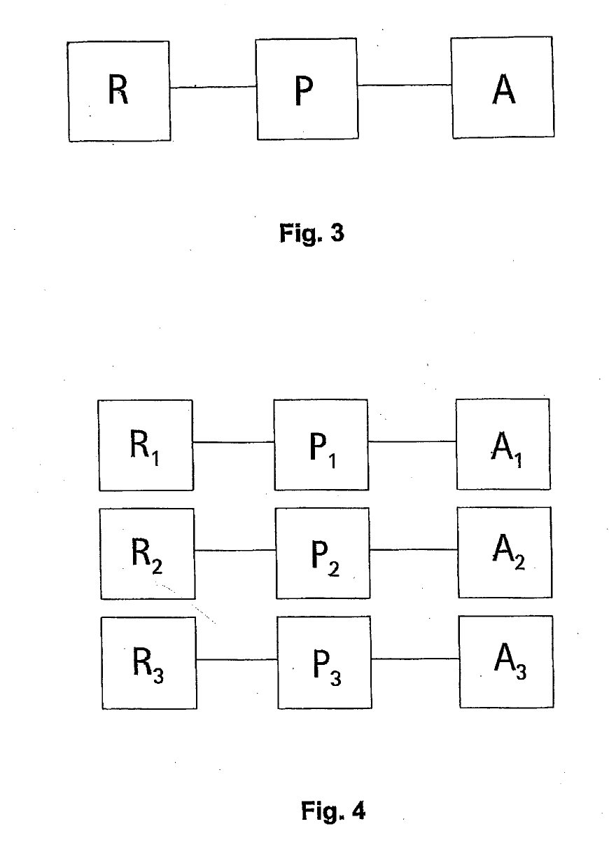

FIG. 3 shows the basic configuration of the device according to the invention with a delivery unit A and a micropump P arranged upstream of the delivery unit which operates as a shutoff and actuation element. The micropump P withdraws a fragrance from a reservoir R. The other elements of the device of the invention, such as the high-voltage unit and controller, are not shown so as not to overcomplicate the drawing.

Unlike the embodiment of FIG. 3, several delivery units A1, A2, A3, and several micropumps P1, P2, P3 and reservoirs R1, R2, R3 can be connected in parallel within a device (FIG. 4). Any number of parallel connections can be implemented in principle.

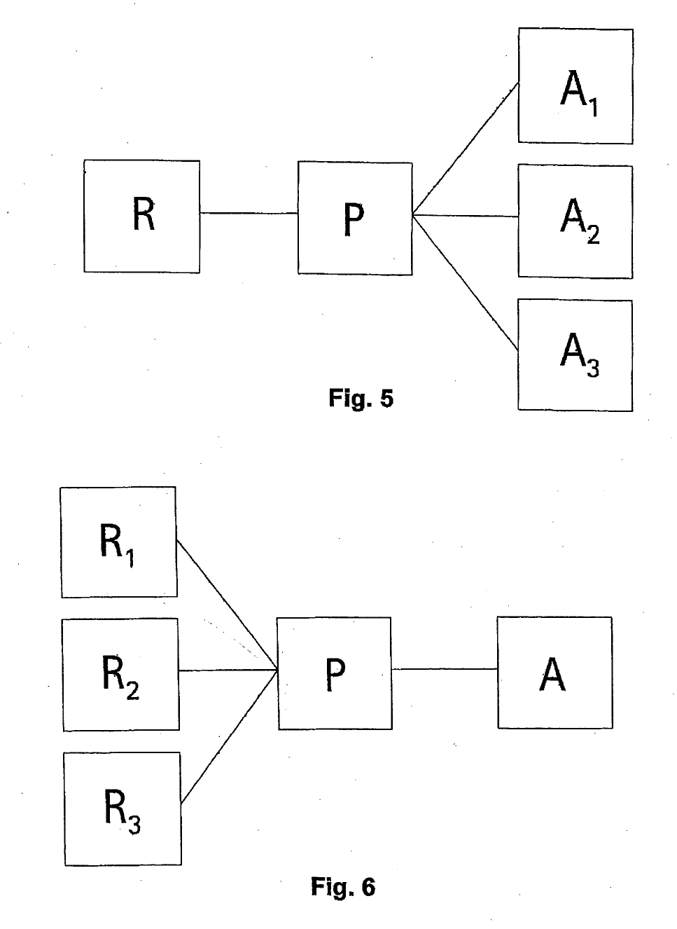

In the embodiment of FIG. 5, several delivery units A1, A2, A3 connected in parallel are supplied from a single reservoir R by a central micropump P, to deliver the fragrance either to different locations, i.e., to separated rooms, or to delivery larger, finely distributed volume flows at a single location.

In the variant depicted in FIG. 6, several reservoirs R1, R2, R3 are connected in parallel. The reservoirs R1, R2, R3 can contain different fragrances. A micropump P withdraws the desired quantity of the fragrance from reservoirs R1, R2, R3 and supplies the fragrance to a central delivery unit A.

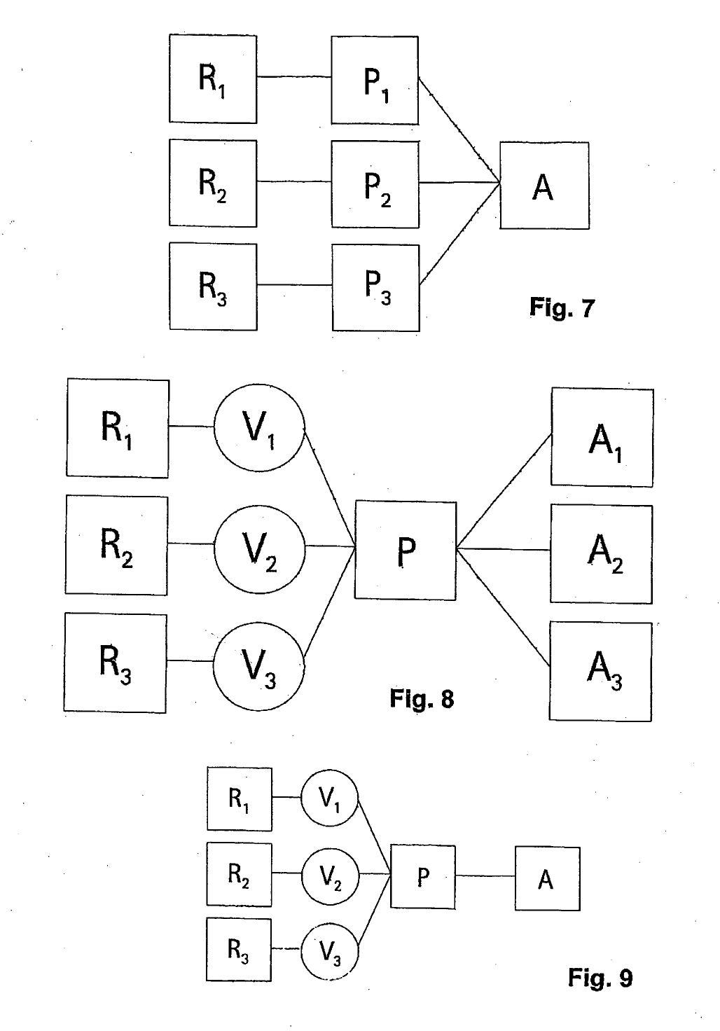

FIG. 7 shows an embodiment with several reservoirs R1, R2, R3 connected in parallel and several micropumps P1, P2, P3 also connected in parallel, which supply the fragrances to a central delivery unit A.

FIG. 8 shows a variant with several reservoirs R1, R2, R3 and several delivery units A1, A2, A3 which are supplied by a central pump P. Unlike in the preceding embodiments, dedicated valves V1, V2, V3 are arranged as a shutoff and actuation elements in each of the supply lines between the individual reservoirs R1, R2, R3 and the central micropump P. The corresponding fragrance can be withdrawn in the desired quantity from the individual reservoirs R1, R2, R3 by opening or closing the supply lines. The valves V are also controlled by a controller, which is not shown in the figure.

FIG. 9 shows a modified embodiment of FIG. 8, wherein the fragrance is delivered to a single central delivery unit A.

A delivery unit in the context of the invention can include one or several small delivery tubes to which a high-voltage is applied, i.e., where a separate electric high voltage is applied to each fragrance. Several delivery units connected in parallel, like those depicted in FIGS. 4, 5 and 8, can also have one or several such delivery tubes.

1. A method for dispensing a liquid fragrance, comprising:
providing a device with
at least one delivery unit,
at least one supply line for supplying the fragrance to the at least one delivery unit,
a high-voltage unit connected to the at least one delivery unit for applying an electric charge to the fragrance for converting the supplied fragrance to an aerosol, and
at least one shutoff and actuating element for shutting off the supply line, and further
activating the at least one shutoff and actuating element and the high-voltage unit with a time offset relative to each other, thereby reducing a quantity of the fragrance disposed inside the supply line between the at least one shutoff and actuating element and the at least one delivery unit.

2. The method according to claim 1, wherein the high-voltage unit is activated before the at least one shutoff and actuating element is opened.

3. The method according to claim 1 and further terminating or interrupting delivery of the fragrance from the at least one delivery unit by first closing the at least one shutoff and actuating element before deactivating the high-voltage unit.

4. The method according to claim 1, wherein the high voltage unit applies a voltage in a range between 0.5 kV and 25 kV to the at least one delivery unit.

5. The method according to claim 1, wherein the high voltage unit applies a voltage in a range between 1.5 kV and 6 kV to the at least one delivery unit.

6. The method according to claim 4, and further maintaining the voltage at a constant level, and controlling the at least one shutoff and actuating element to adjust a volume flow rate of the fragrance.

7. The method according to claim 6, wherein the volume flow rate is adjusted by controlling a micropump.

8. The method according to claim 7, wherein the volume flow rate of the micropump is adjusted to have a maximum value that is smaller than or equal to a delivery capacity of the at least one delivery unit.

9. The method according to claim 1, wherein different ones of the at least one delivery unit are supplied with a different fragrance, and wherein the different fragrances are separately converted into aerosols by separately controlling the time offset.

10. The method according to claim 1, and further withdrawing the fragrance via the at least one supply line from an exchangeable fragrance reservoir having a flexible casing.

11. A device for dispensing a liquid fragrance, comprising:
at least one delivery unit,
at least one supply line for supplying the fragrance to the at least one delivery unit,
a high-voltage unit connected to the at least one delivery unit for applying an electric charge to the fragrance for converting the supplied fragrance to an aerosol,
at least one shutoff and actuating element for shutting off the supply line,
a micropump for adjusting a volume flow rate of the fragrance, said micropump having a maximum delivery volume that is smaller than a maximum delivery capacity of the at least one delivery unit, and
a controller for controlling the at least one shutoff and actuating element or the micropump, or a combination thereof,
wherein the controller activates the high-voltage unit and the micropump with a time offset so that as to reduce an amount of the fragrance disposed inside the at least one supply line between the at least one shutoff and actuating element and the at least one delivery unit (4, A).

12. The method according to claim 6, wherein different ones of the at least one delivery unit are supplied with a different fragrance, and wherein the different fragrances are separately converted into aerosols by separately controlling the volume flow rate.

13. The device of claim 11, further comprising a plurality of reservoirs, each reservoir connected to the at least one delivery unit by a dedicated micropump, with the controller controlling the dedicated micropumps.

14. The device of claim 13, wherein the reservoirs are exchangeable and have a flexible casing.

15. The device of claim 11, further comprising a reservoir containing the fragrance, wherein the reservoir is exchangeable and has a flexible casing.

16. The device of claim 11, wherein the high voltage unit supplies a voltage in a range between 0.5 kV and 25 kV.

17. The device of claim 11, wherein the high voltage unit supplies a voltage in a range between 1.5 kV and 6 kV.
